(19) United States
(12) Patent Application Publication
(10) Pub. No.: US 2026/0290612 A1
(43) Pub. Date: Sep. 24, 2026

(54) SYSTEMS AND METHODS TO MEASURE AND INFER DIGITAL BIOMARKERS INTO HEALTH INSIGHTS

(71) Applicant: BioTrillion, Inc., San Francisco, CA (US)

(72) Inventors: Savan R. Devani, San Francisco, CA (US); Derrick Mo, San Francisco, CA (US)

(21) Appl. No.: 19/569,934

(22) Filed: Mar. 17, 2026

Related U.S. Application Data
(60) Provisional application No. 63/773,751, filed on Mar. 18, 2025.

(51) Int. Cl. G16H 50/30; G16H 10/60

(52) U.S. Cl. CPC G16H 50/30; G16H 10/60

(57) ABSTRACT

A digital biomarker system uses sensor data captured by a user device and processed on the user device, by one or more remote computing systems, or in a hybrid combination thereof to generate health insights associated with a user. Sensor data for at least one capture window are used to determine signal-acquisition condition metrics and perform threshold-based quality gating. Responsive to threshold satisfaction, accepted data are preprocessed and used to extract digital biomarker signs. Sign-state data are generated for one or more signs, including a status and at least one of a trend, an inflection, a confidence value, or a dynamic weighting value. Rule-out and rule-in analysis are performed using at least the sign-state data to determine and rank one or more health insights. The system can optionally use contextual covariates or adaptive symptom question responses and can optionally generate a composite health score and subsystem scores.

### CROSS-REFERENCE TO RELATED APPLICATION

This application claims the benefit of and priority to U.S. Provisional Patent Application No. 63/773,751, filed Mar. 18, 2025, which is hereby incorporated by reference herein in its entirety.

### FIELD OF THE DISCLOSURE

The present disclosure relates generally to digital health sensing, inference, and monitoring, and more particularly to systems and methods for capturing sensor data, determining technical capture quality, extracting Digital Biomarker Signs, generating Sign-State Data including Status and longitudinal characteristics, and determining ranked Health Insights using on-device processing, cloud processing, or hybrid processing.

### BACKGROUND

Many digital health tools focus on a single biomarker or a single sensing modality and can become brittle under variable capture conditions, unstable when data quality is poor, or difficult to scale outside specialized environments.

Other approaches emphasize questionnaire-only symptom collection, isolated abnormal readings, or opaque scoring approaches that do not explicitly incorporate reassuring evidence, longitudinal Trends, confidence gating, or structured Rule-Out and Rule-In reasoning.

Existing approaches also frequently underutilize the sensing capabilities available on modern user devices and related computing environments. Commodity devices can include optical sensors, inertial sensors, microphones, thermal or depth-capable sensors, environmental sensors, and other contextual inputs, but many systems do not effectively combine these capabilities in a unified measurement-and-interpretation pipeline.

In many existing systems, measurement acquisition and downstream interpretation are treated as separate problems. A reading may be accepted without sufficiently evaluating whether the capture conditions were suitable for reliable extraction or inference. Poor lighting, poor framing, excessive motion, transient occlusion, audio contamination, or other acquisition defects may therefore propagate into reduced reliability, false alerts, or low-value outputs.

Existing approaches also often fail to distinguish between technical capture-suitability information and health-interpretation information. A first category relates to technical capture suitability, such as lighting adequacy, framing quality, motion artifact, focus quality, occlusion, or other Signal-Acquisition Condition Metrics. A second category relates to health interpretation, such as demographic attributes, behavioral context, historical context, environmental influences, and other physiologically relevant covariates. Failure to separate these categories can reduce interpretability and system robustness.

Another limitation of many existing systems is that they rely primarily on single-session outputs. Longitudinal Trends, Trend reversals, Inflections, baseline shifts, and changes in multi-Sign patterns over time may carry clinically or operationally meaningful information that is not captured by static single-session analysis.

A further limitation is that many current systems do not effectively combine objective measurement with a constrained and targeted user dialogue. A general symptom questionnaire may be too long, too generic, or poorly tied to measured abnormalities. Conversely, a measurement-only system may omit useful contextual clarification that could improve ranking, explanation, or prioritization of candidate Health Insights.

Existing systems may also present results in only one format, such as a simplified user-facing output or a specialized technical display, and may not effectively support multiple renderings from a common underlying inference result.

There therefore remains a need for systems and methods that can capture multimodal source data using commodity devices, determine whether such data are of sufficient quality, extract multiple Digital Biomarker Signs, convert such Signs into inference-ready Sign-State Data, combine such Sign-State Data with optional contextual covariates and optional Adaptive Symptom Questions, and generate ranked Health Insights, scores, and related outputs in a manner suitable for on-device, cloud, and hybrid implementations.

### SUMMARY

In some implementations, a system obtains sensor data for at least one Capture Window using at least one sensor of a User Device. The sensor data can include optical data, kinetic data, acoustic data, behavioral data, thermal data, depth-related data, environmental sensor data, or other sensor-derived data, and can be obtained from one modality or from multiple modalities.

In some implementations, the system outputs one or more instructions during a pre-capture stage and/or during the Capture Window to guide performance of one or more user activities that facilitate calibration, alignment, signal conditioning, or computation of one or more metrics. Such user activities can include positioning, holding still, changing gaze, opening or closing the eyes, walking, speaking, breathing, or repositioning the User Device. Guidance can include, for example, a silhouette overlay, face-mesh guidance, alignment indicators, and/or haptic confirmation when one or more gating conditions are satisfied.

In some implementations, the system determines one or more Signal-Acquisition Condition Metrics characterizing technical capture quality and performs Quality Gating by determining whether one or more thresholds are satisfied. When one or more thresholds are not satisfied, the system can initiate one or more capture-remediation actions such as guidance, recapture, partial recapture, segment discarding, selective suppression, and/or confidence-based down-weighting of one or more Signs during downstream processing. The Signal-Acquisition Condition Metrics can include, by way of example, lighting adequacy, exposure balance, framing quality, motion contamination, blur, focus quality, occlusion, audio quality, signal-to-noise sufficiency, and physiologic plausibility.

In some implementations, responsive to threshold satisfaction, the system preprocesses accepted sensor data and extracts a plurality of Digital Biomarker Signs from the accepted sensor data. The extracted Signs can include Signs derived from optical processing, motion processing, audio processing, thermal processing, depth processing, behavioral processing, or combinations thereof.

In some implementations, the system generates Sign-State Data for one or more Signs. The Sign-State Data can include a Status and at least one of a Trend, an Inflection, a Confidence value, a Dynamic Weighting value, or any combination thereof. In some implementations, the Sign-State representation supports a Status-and-Trends framework in which each Sign is characterized by a single-session Status and, when longitudinal data are available, a Trend across repeated sessions or observation windows.

In some implementations, the system assembles an inference input that includes at least the Sign-State Data and, when available, zero or more contextual covariates. The contextual covariates can include demographic attributes, behavioral context, historical context, Health-Relevant Environmental Covariates, or combinations thereof. Health-Relevant Environmental Covariates can include external factors that influence physiology or interpretation, whereas the Signal-Acquisition Condition Metrics relate to technical data suitability.

In some implementations, a Combinatorial Inference Engine performs Rule-Out analysis and Rule-In analysis using at least the Sign-State Data, and optionally using contextual covariates and/or responses to a limited sequence of Adaptive Symptom Questions, to determine and rank one or more Health Insights associated with the user. The Health Insights can span wellness, exposure, symptom-related, condition-pattern, disease-risk, progression, recovery, performance, early-detection, and related human-health outputs, while remaining non-diagnostic and non-treatment-oriented.

In some implementations, the Combinatorial Inference Engine applies plausibility constraints, contradiction handling, cross-domain corroboration, Trend-based refinement, and machine-learning-assisted mapping. In some implementations, ranking can be based on one or more of confidence, specificity, sensitivity, contextual fit, contradiction resolution, and temporal stability.

In some implementations, the system optionally refines inference using a limited sequence of Adaptive Symptom Questions and/or optionally produces a Composite HealthScore and one or more Subsystem Scores using smoothing, hysteresis, weighting, penalty logic, or combinations thereof. The Composite HealthScore is not required for operation of the Combinatorial Inference Engine and can be omitted. The same underlying result can be rendered as a user-facing summary, a clinician-style summary, a structured output, an export package, or another rendering.

In some implementations, the disclosed functionality can execute on the User Device, in the cloud, or in a hybrid combination of the two. Thus, sensor data can be captured by the User Device and then processed, interpreted, ranked, explained, or routed on-device, in one or more remote computing systems, or in distributed fashion across both.

In some implementations, sensor data and/or derived Sign data can additionally be obtained across one or more Capture Windows, one or more observation windows, one or more modalities, and/or one or more cooperating devices, while still being consolidated into a common measurement-and-interpretation pipeline.

In some implementations, optional privacy-aware processing can include selective transmission of derived data rather than raw data, secure identifiers, pseudonymous or de-identified remote refinement, access controls, retention controls, deletion workflows, and auditability features. Such privacy or governance features can be implemented when desired and are not required in all implementations.

### BRIEF DESCRIPTION OF THE DRAWINGS

FIG. 1 is a block diagram illustrating an example system architecture including a User Device, an on-device processing path, and an optional cloud processing path;

FIG. 2 is a flow diagram illustrating an example measurement-layer method including pre-capture guidance, sensor-data acquisition, Signal-Acquisition Condition Metric determination, threshold-based Quality Gating, preprocessing, and Digital Biomarker Sign extraction;

FIG. 3 is a flow diagram illustrating an example Sign-State generation and interpretation-layer method including generation of Sign-State Data, optional contextual fusion, Rule-Out analysis, Rule-In analysis, ranking, and output generation;

FIG. 4 is a flow diagram illustrating an example Quality Gating subroutine including technical capture-quality determination, threshold testing, acceptance, remediation, and downstream confidence propagation;

FIG. 5A illustrates representative user-interface views including a capture-guidance view;

FIG. 5B illustrates a measurement view showing quantified Signs;

FIG. 5C illustrates a score-summary view;

FIG. 5D illustrates an Adaptive Symptom Question view; and

FIG. 5E illustrates a ranked Health Insight view.

### DETAILED DESCRIPTION

The present disclosure will now be described with reference to illustrative implementations. Features described in connection with one implementation can be used in other implementations unless context indicates otherwise.

As used herein, a “User Device” refers to a device that includes one or more sensors capable of generating sensor data for use by the present disclosure. A User Device can be, for example, a smartphone, tablet, wearable-assisted device, laptop, workstation-connected camera device, web-camera-equipped device, or another portable or non-portable computing device.

As used herein, a “Capture Window” is a defined time interval over which one or more modality data streams are collected for an assessment session. A Capture Window can include all or part of a facial-video session, a body-video session, a motion session, an acoustic session, a guided user-activity session, a stimulus-evoked session, or another sensor-acquisition session. In some implementations, different modalities can be captured synchronously within the same Capture Window. In other implementations, different modalities can be captured asynchronously across multiple Capture Windows or observation windows and later consolidated.

As used herein, “Signal-Acquisition Condition Metrics” refer to technical capture-quality metrics used to determine whether sensor data are suitable for downstream analysis. Examples can include lighting adequacy, exposure balance, framing quality, face or body visibility, motion artifact, blur, focus quality, occlusion, audio quality, signal-to-noise sufficiency, and physiologic plausibility.

As used herein, “Digital Biomarker Sign” or “Sign” refers to a quantified indicator derived from sensor data. A Sign can be a raw-derived value, normalized value, percentile value, categorical value, score, or other quantified representation derived from one or more accepted data segments.

As used herein, “Status” refers to a single-session state of a Sign for a current Capture Window or current observation window. As used herein, “Trend” refers to longitudinal behavior over time of that same Sign across repeated sessions or observation windows. As used herein, “Inflection” refers to a reversal, threshold crossing, or other directional change in a Trend.

As used herein, “Health-Relevant Environmental Covariates” refer to context variables that may influence physiology or interpretation, such as air quality, heat index, ultraviolet exposure, temperature, humidity, circadian context, time-of-day context, or other external factors. Such covariates are distinct from the Signal-Acquisition Condition Metrics. In some implementations, such environmental information can be directly sensed using device sensors. In other implementations, it can be obtained from user input, local modules, network-accessed services, or combinations thereof.

As used herein, “Health Insight” refers broadly to a non-diagnostic output associated with human health and can include a wellness insight, exposure insight, symptom-related insight, condition-pattern insight, disease-risk insight, progression-related insight, recovery-related insight, performance-related insight, early-detection insight, or another health-related output.

As used herein, “Composite HealthScore” refers to an aggregate score computed from a set of Sign-State inputs and optionally one or more Subsystem Scores. In certain implementations, the Composite HealthScore can be presented under a product label such as “HealthScore,” and the Subsystem Scores can be presented under implementation-specific labels such as cardio, neuro, mood, mobility, energy, or stress.

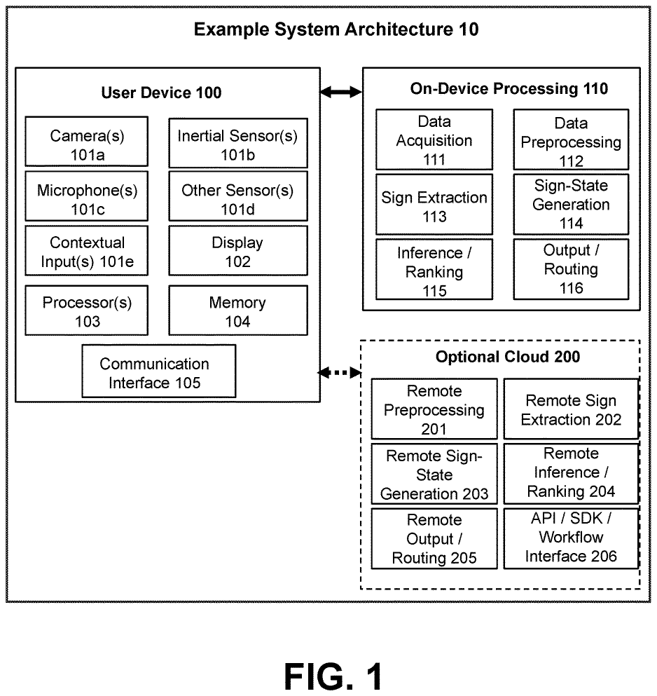

FIG. 1 illustrates system 10 including a User Device 100, an on-device processing path 110, and an optional cloud processing path 200. The architecture of FIG. 1 is intended to show that sensor data are generated at the User Device 100 and then processed on-device, in the cloud, or in a hybrid combination of the two.

In the illustrated implementation, the User Device 100 includes one or more Cameras 101a, one or more Inertial Sensors 101b such as accelerometers and gyroscopes, one or more Microphones 101c, optionally one or more Other Sensors 101d such as thermal sensors, depth-capable sensors, or environmental sensors, one or more Contextual Inputs 101e, a Display 102, one or more Processors 103, Memory 104, and a Communication Interface 105.

The Contextual Inputs 101e can include user-entered information, stored profile information, historical session data, network-accessed contextual data, directly sensed environmental data, or combinations thereof.

The on-device processing path 110 can include, for example, a Data Acquisition function 111, a Data Preprocessing function 112, a Sign Extraction function 113, a Sign-State Generation function 114, an Inference/Ranking function 115, and an Output/Routing function 116. These functions can be implemented in software, firmware, hardware, or combinations thereof.

The optional cloud processing path 200 can include, for example, a Remote Preprocessing function 201, a Remote Sign Extraction function 202, a Remote Sign-State Generation function 203, a Remote Inference/Ranking function 204, a Remote Output / Routing function 205, and optionally one or more API/SDK or workflow interfaces 206. The cloud processing path 200 is optional and can be illustrated in dotted lines in the drawings.

In some implementations, the on-device processing path 110 and the cloud processing path 200 perform analogous functions at different execution locations. Thus, preprocessing, Sign extraction, Sign-State generation, inference, ranking, explanation generation, score generation, summary generation, routing, or export can occur on-device, in the cloud, or in hybrid fashion. A result determined remotely can be returned to the User Device 100, routed to another destination, or both.

In some implementations, the same architecture can additionally accept data or derived Signs from one or more cooperating devices, provided that such information is consolidated into the same downstream Sign-State and interpretation pipeline. Examples of cooperating devices can include wearables, hearables, connected cameras, smart mirrors, connected home devices, or other sensor-enabled devices.

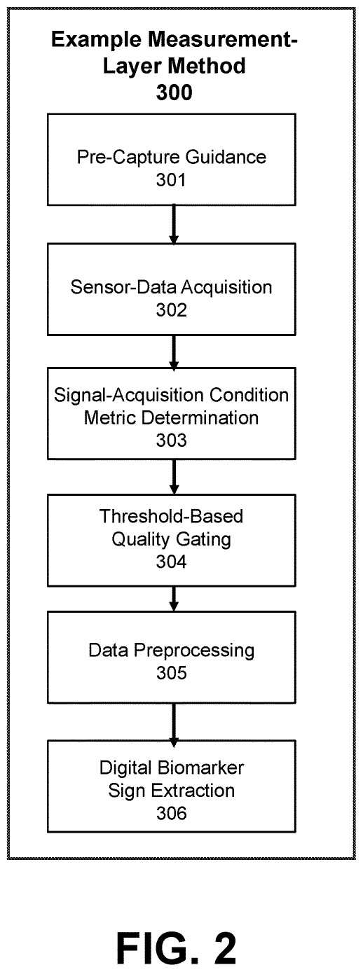

FIG. 2 illustrates an example measurement-layer method 300. Method 300 can include Pre-Capture Guidance 301, Sensor-Data Acquisition 302, Signal-Acquisition Condition Metric Determination 303, Threshold-Based Quality Gating 304, Data Preprocessing 305, and Digital Biomarker Sign Extraction 306.

During Pre-Capture Guidance 301, the system can present visual guidance, audio guidance, haptic guidance, or combinations thereof to improve capture suitability. Such guidance can include augmented-reality overlays, silhouette guidance, face-mesh guidance, face-position indicators, body-position indicators, distance guidance, angle guidance, and prompts to improve lighting, reduce motion, improve framing, or prepare for a guided user activity.

In some implementations, the system can output one or more instructions before or during acquisition to guide performance of one or more user activities or stimuli that facilitate calibration, alignment, signal conditioning, or computation of one or more metrics. Such activities can include holding still, repositioning the device, changing gaze, opening or closing the eyes, walking, speaking, breathing, or changing body position. In some implementations, a controlled display-luminance change or other optical stimulus can be used during a Capture Window to improve extraction of pupil-response-related Signs. In some implementations, a moving target can be displayed to elicit gaze-related or oculomotor responses.

During Sensor-Data Acquisition 302, the system can acquire optical data, kinetic data, acoustic data, behavioral data, thermal data, depth-related data, environmental data, and/or other supported modality data during one or more Capture Windows or observation windows. Optical data can be acquired using a front-facing camera, a rear-facing camera, or another optical sensor. Kinetic data can be acquired using inertial sensors. Acoustic data can be acquired using microphones.

In some implementations, the sensor data can be captured synchronously. In other implementations, one modality can be captured during a shorter first Capture Window while another modality is captured during a longer second observation window, and the resulting data can later be consolidated. Thus, the present disclosure is not limited to strictly synchronous multimodal acquisition.

Signal-Acquisition Condition Metric Determination 303 can be performed before, during, and/or after acquisition. The system can determine one or more technical capture-quality values from the sensor data and/or associated metadata. For example, the system can determine lighting adequacy from luminance or histogram information, framing quality from region-of-interest detection, motion contamination from image-frame displacement or inertial movement values, blur from focus metrics, audio quality from noise-floor or signal-to-noise measurements, and physiologic plausibility from whether an extracted candidate signal falls within one or more expected bands or patterns.

Threshold-Based Quality Gating 304 can include determining whether one or more Signal-Acquisition Condition Metrics satisfy one or more thresholds. If one or more thresholds are not satisfied, the system can prompt recapture, partial recapture, repositioning, improved lighting, reduced motion, or another corrective action. In some implementations, only a corrupted segment is replaced rather than requiring a full restart.

If one or more thresholds are satisfied, or if a particular data segment is accepted with a lower confidence designation, the system can proceed to Data Preprocessing 305. In some implementations, certain data can be suppressed entirely, while other data can be retained but down-weighted.

Data Preprocessing 305 can include one or more modality-specific or cross-modality operations. For optical data, preprocessing can include frame stabilization, noise reduction, ambient-light or luminance calibration, histogram-based or brightness-based correction, region-of-interest tracking, color-space transformation, artifact rejection, temporal segmentation, and spectral transforms. Example color spaces can include YUV, YCrCb, and CIELAB. Example transforms can include band-pass filtering and frequency-domain analysis.

For kinetic data, preprocessing can include filtering, root-mean-square normalization, frequency-domain analysis, downsampling, gravity-component removal, event detection, and motion-specific artifact rejection. For depth-related data, preprocessing can include depth-frame denoising, smoothing, contour stabilization, or reference-surface stabilization. For acoustic data, preprocessing can include filtering, segmentation, noise suppression, and temporal or spectral analysis.

Digital Biomarker Sign Extraction 306 can include multiple modality-specific or cross-modality extraction pipelines. Optical extraction can include, by way of example, remote photoplethysmographic extraction, colorimetric extraction, respiratory extraction, pupil-related extraction, eyelid-related extraction, oculomotor extraction, facial asymmetry extraction, edema extraction, facial geometry extraction, skin-related extraction, lip-color extraction, scleral-color extraction, or related visual-sign extraction.

Kinetic extraction can include walking speed, step length, cadence, double support time, gait variability, asymmetry, tremor-related movement, balance-related movement, recovery-related movement, fatigue-related movement, or other mobility-related Signs. Acoustic extraction can include speech, cough, breathing, vocal tremor, hesitation, pacing, respiratory acoustics, or other audio-derived Signs. Behavioral extraction can include task-completion behavior, interaction timing, adherence signals, or movement behavior.

The architecture remains extensible to future sensors and future sensor-derived primitives without changing the core pipeline. New modalities can be added as new Sign sources, contextual sources, corroborative sources, or confidence-modulating sources.

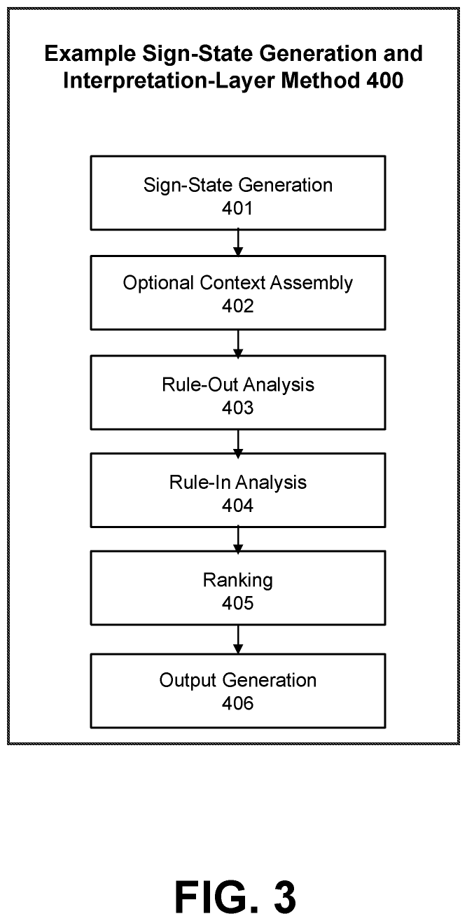

FIG. 3 illustrates an example interpretation-layer method 400. Method 400 can include Sign-State Generation 401, Optional Context Assembly 402, Rule-Out Analysis 403, Rule-In Analysis 404, Ranking 405, and Output Generation 406.

Sign-State Generation 401 converts extracted Sign values into richer representations used for downstream inference. This can include normalization relative to a reference population, a personal baseline, prior sessions, contextual covariates, or combinations thereof. It can further include determination of a Status, a Trend, an Inflection, a Confidence value, a Dynamic Weighting value, or any combination thereof.

In some implementations, Status is derived by comparing an extracted Sign value to one or more ranges, thresholds, percentiles, or other decision boundaries. In some implementations, Trend is derived from longitudinal comparison across repeated sessions or observation windows. In some implementations, Inflection is derived by identifying a reversal, threshold crossing, or regime change in a Trend. In some implementations, Confidence is derived from one or more Signal-Acquisition Condition Metrics, cross-validation checks, plausibility checks, model stability checks, or corroboration across modalities.

Although certain implementations can use a Low/Average/High Status framework and a Decreasing/Stable/Increasing Trend framework, the present disclosure is not limited to exactly three Status states or exactly three Trend states. Additional state taxonomies can be used.

Optional Context Assembly 402 can include forming an inference input from the Sign-State Data and, when available, zero or more contextual covariates. The contextual covariates can include demographic attributes such as age, sex, or body-composition indicators, behavioral context, historical context, medication context, activity context, recovery context, Health-Relevant Environmental Covariates, or combinations thereof.

Rule-Out Analysis 403 can use normal, average, reassuring, stable, improving, low-risk, or otherwise inconsistent Sign-State evidence to reduce support for one or more possible Health Insights. Rule-In Analysis 404 can use high, low, abnormal, worsening, corroborative, or otherwise consistent Sign-State evidence to increase support for one or more Health Insights.

By incorporating both reassuring and concerning evidence, the disclosed Rule-Out/Rule-In framework can improve specificity relative to approaches that consider only abnormal findings. The framework can also reduce over-weighting of isolated abnormal values when broader multi-Sign evidence is inconsistent with a particular Health Insight.

In some implementations, the Combinatorial Inference Engine can apply plausibility constraints, contradiction handling, cross-domain corroboration, Trend-based refinement, and machine-learning-assisted mapping to determine and prioritize one or more Health Insights. In some implementations, the engine can be updated using historical outcomes, external data, user history, remote refinement, or other feedback sources.

Ranking 405 can rank one or more Health Insights based on one or more of confidence, specificity, sensitivity, contextual fit, contradiction resolution, temporal stability, or combinations thereof. In some implementations, the system outputs a top-ranked subset, such as one, two, three, or more ranked Health Insights.

In some implementations, a limited sequence of Adaptive Symptom Questions can optionally be introduced before final ranking or between preliminary ranking and final ranking. The Adaptive Symptom Questions can be selected from a predefined question bank, a dynamically generated question set, a retrieval-supported question source, or a hybrid question source, provided that the questions are constrained by measured Signs, Sign-State Data, contextual covariates, preliminary Health Insights, or combinations thereof.

In some implementations, the limited sequence of Adaptive Symptom Questions comprises exactly three Adaptive Symptom Questions. A three-question implementation can include, for example, a symptom-clarification question, a temporal or frequency clarification question, and a severity, recovery, or capacity clarification question. Other limited sequences can also be used.

In some implementations, responses to Adaptive Symptom Questions can confirm, discount, suppress, or reprioritize one or more Health Insights.

Output Generation 406 can include user-facing outputs, clinician-style outputs, structured outputs, workflow outputs, export packages, or combinations thereof. Such outputs can include simplified explanations, trend summaries, score panels, alerts, rationale summaries, structured fields, Rule-Out observations, Rule-In observations, ranked Health Insight presentations, or combinations thereof.

The same underlying Sign-State Data and Health Insight result can be rendered into more than one output form without changing the underlying measurement or inference pipeline. For example, a first rendering can be a simplified user-facing summary and a second rendering can be a clinician-style summary or structured output emphasizing rationale, longitudinal context, or ranked observations.

In some implementations, the system additionally computes a Composite HealthScore and optionally one or more Subsystem Scores from Sign-State Data. The Subsystem Scores can correspond to domains such as cardio, neuro, mood, mobility, energy, stress, or other domains. The taxonomy is not fixed and can vary by implementation.

The Composite HealthScore and Subsystem Scores can be computed using weighted aggregation, smoothing, hysteresis, penalty functions, confidence weighting, domain-specific rules, or combinations thereof. Such scores can be used for trend tracking, summarization, alerting, segmentation, or presentation, and can be provided in addition to or independently from ranked Health Insights.

Repeated sessions over time can support Derived Longitudinal Indices, including fatigue-related, mood-related, pain-related, diurnal-asymmetry, condition-day, or other longitudinal indices. These are examples only and are not required in all implementations.

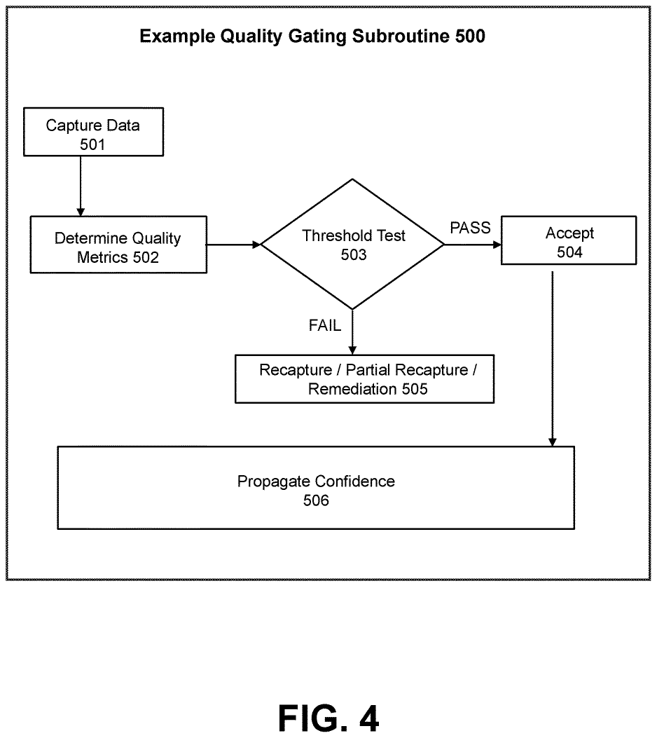

FIG. 4 illustrates an example Quality Gating subroutine 500. Capture Data 501 are received, and one or more Quality Metrics 502 are determined. Threshold Test 503 determines whether one or more technical capture criteria are satisfied. If Threshold Test 503 passes, the data are accepted at 504 for downstream use. If Threshold Test 503 fails, Recapture/Partial Recapture/Remediation 505 can be requested. Confidence Propagation 506 then passes quality information into downstream weighting, Sign-State generation, ranking, score generation, and output generation.

In some implementations, contradiction checks and physiologic-plausibility checks can be applied before, during, or after Threshold Test 503. In some implementations, a Sign can remain available for downstream use even if one or more condition metrics are suboptimal, provided that the Sign is down-weighted or annotated with lower confidence. In other implementations, one or more Signs can be suppressed entirely.

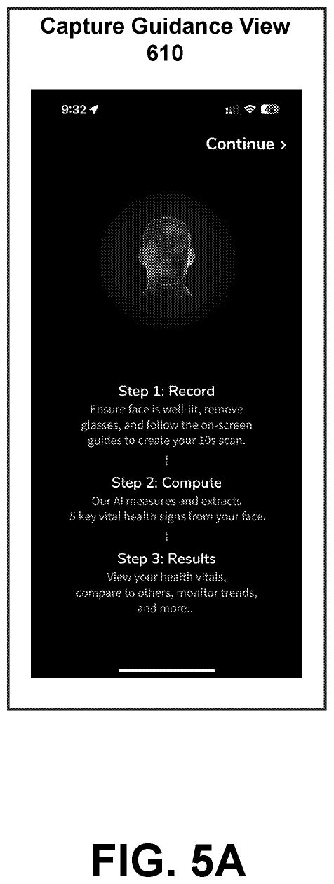

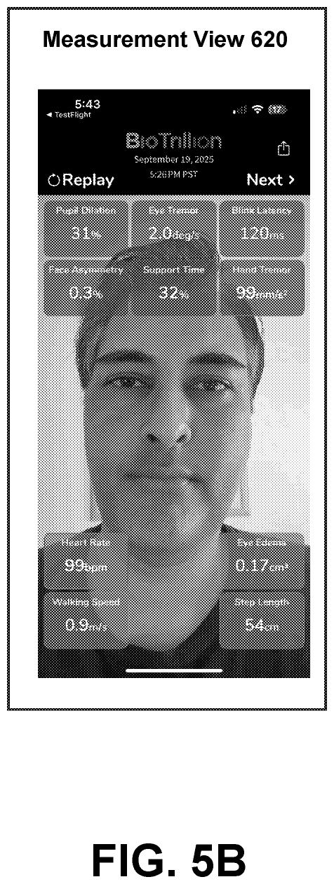

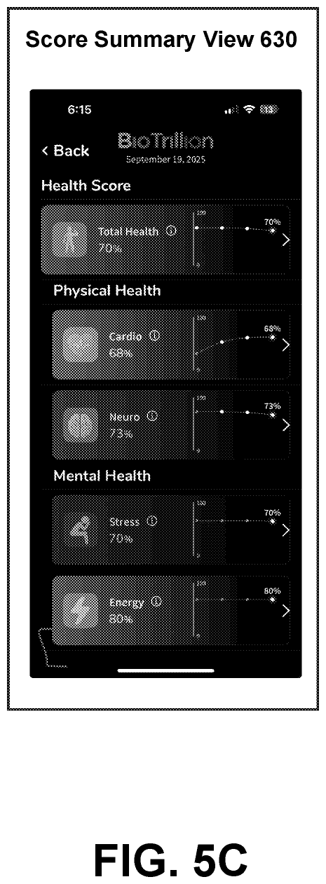

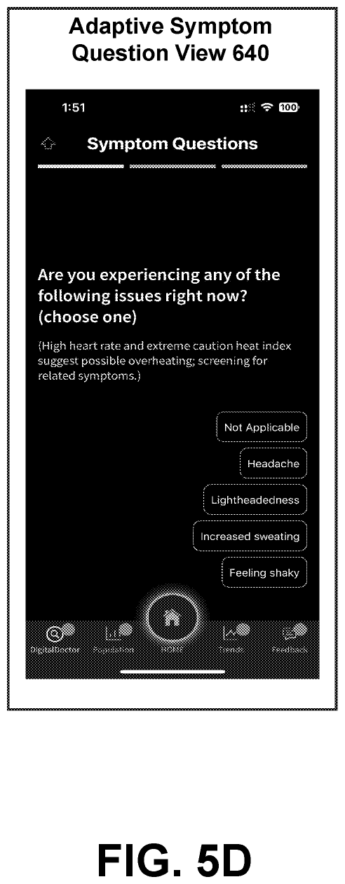

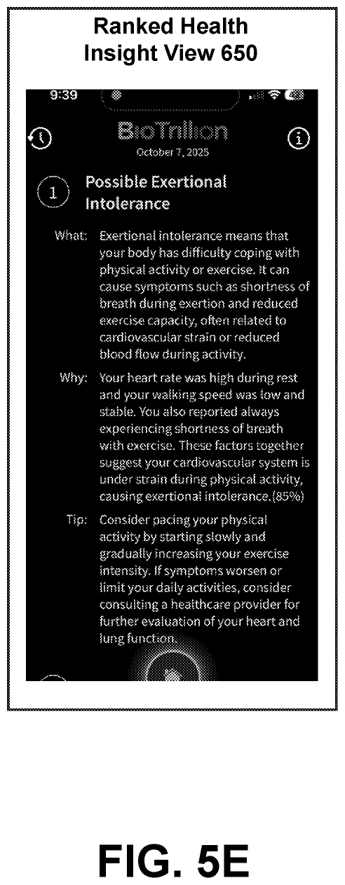

FIGS. 5A-5E collectively illustrate representative user-interface views. In one implementation, FIG. 5A illustrates a capture-guidance view 610 that instructs the user how to position the face or device for a recording. FIG. 5B illustrates a measurement view 620 that can display quantified Signs obtained from an accepted capture. FIG. 5C illustrates a score-summary view 630 that can display a Composite HealthScore and one or more Subsystem Scores. FIG. 5D illustrates an Adaptive Symptom Question view 640 that can present a targeted question and response options. FIG. 5E illustrates a ranked Health Insight view 650 that can present at least one ranked Health Insight together with explanatory content.

The user-interface views of FIGS. 5A-5E are illustrative only and can be simplified or reformatted. In some implementations, the output is rendered on the same User Device used for capture. In some implementations, the output is determined remotely and returned to the User Device. In some implementations, the output is additionally routed to another destination.

In some implementations, replay or auditability features can also be provided, including display or storage of a captured session with overlays such as face meshes, landmarks, or highlighted regions to illustrate what was measured or why a Sign was accepted, down-weighted, or rejected.

Optional privacy or governance features can include selective transmission of raw or derived data, user permissions, secure identifiers, de-identified remote refinement, retention controls, deletion controls, access logging, audit trails, or related mechanisms. These features can be implemented when desired and are not required in all implementations.

Although various implementations have been described herein, numerous modifications and variations will be apparent to people who have ordinary skill in the art in view of the present disclosure. Such modifications and variations are intended to fall within the scope of the appended claims.

What is claimed is:

1. A computer-implemented method for generating one or more health insights associated with a user, the method comprising:
obtaining, using at least one sensor of a user device during at least one capture window, sensor data comprising data from at least one modality;
determining, based at least in part on the sensor data, one or more signal-acquisition condition metrics characterizing technical capture quality;
determining whether the one or more signal-acquisition condition metrics satisfy one or more thresholds;
responsive to determining that the one or more thresholds are satisfied, preprocessing at least a portion of the sensor data and extracting a plurality of digital biomarker signs from the sensor data;
generating, for at least one of the plurality of digital biomarker signs, sign-state data comprising a status and at least one of a trend, an inflection, a confidence value, a dynamic weighting value, or any combination thereof;
performing rule-out analysis and rule-in analysis using at least the sign-state data to determine one or more health insights associated with the user;
rank-ordering the one or more health insights; and
outputting at least one ranked health insight.

2. The method of claim 1, wherein the sensor data comprises data from one or more modalities selected from optical, kinetic, acoustic, behavioral, thermal, depth-related, environmental, and other sensor-derived modalities.

3. The method of claim 1, wherein the one or more signal-acquisition condition metrics comprise at least one metric selected from lighting adequacy, exposure balance, framing quality, face visibility, body visibility, motion artifact, blur, focus quality, occlusion, audio quality, signal-to-noise sufficiency, and physiologic plausibility.

4. The method of claim 1, wherein responsive to determining that the one or more thresholds are not satisfied, the method further comprises performing one or more remediation operations selected from recapture, partial recapture, repositioning guidance, lighting guidance, stabilization guidance, region-of-interest guidance, selective suppression of unreliable signs, and confidence-based down-weighting of unreliable signs.

5. The method of claim 1, further comprising determining one or more contextual covariates and using the one or more contextual covariates together with the sign-state data to determine the one or more health insights, wherein the one or more contextual covariates comprise one or more values selected from demographic attributes, behavioral context, historical context, health-relevant environmental covariates, and combinations thereof.

6. The method of claim 1, further comprising normalizing at least one digital biomarker sign based on a contextual covariate, a personal baseline, a reference population, a prior session, or any combination thereof, and applying dynamic weighting based on the status, the trend, the confidence value, or any combination thereof.

7. The method of claim 1, further comprising selecting, based on at least the sign-state data, a limited sequence of adaptive symptom questions, receiving one or more responses to the limited sequence of adaptive symptom questions, and refining at least one of the rule-out analysis, the rule-in analysis, or the rank-ordering based on the one or more responses.

8. The method of claim 7, wherein the limited sequence of adaptive symptom questions comprises one or more adaptive symptom questions including a symptom-clarification question, a temporal or frequency clarification question, and a severity, recovery, or capacity clarification question.

9. The method of claim 7, wherein the rank-ordering is based on one or more factors selected from confidence, specificity, sensitivity, contextual fit, contradiction resolution, temporal stability, and combinations thereof.

10. The method of claim 1, further comprising, during a pre-capture stage and/or during the at least one capture window, outputting one or more instructions configured to guide performance of one or more user activities that facilitate calibration, alignment, signal conditioning, or computation of one or more metrics.

11. The method of claim 10, wherein the one or more user activities comprises holding still, repositioning the user device, changing gaze, opening or closing eyes of the user, walking, speaking, breathing, changing body position, or any combination thereof.

12. The method of claim 1, further comprising computing, from at least the sign-state data, a composite health score, and one or more subsystem scores using smoothing, hysteresis, weighted aggregation, penalty logic, or any combinations thereof.

13. The method of claim 1, wherein at least a first portion of the sensor data is obtained during a first capture window from the user device and at least a second portion of sensor data is obtained during a second observation window from the user device or a cooperating device, and wherein the one or more health insights are determined using digital biomarker signs derived from both the first portion and the second portion.

14. The method of claim 1, wherein the one or more ranked health insights comprise at least one of wellness insights, exposure insights, symptom-related insights, condition-pattern insights, disease-risk insights, progression-related insights, recovery-related insights, performance-related insights, and early-detection insights.

15. A system for generating one or more health insights associated with a user, the system comprising:
a user device comprising at least one sensor, a display, one or more processors, memory, and a communication interface; and
instructions stored in the memory and executable by the one or more processors to cause the system to:
obtain, using the at least one sensor, sensor data during at least one capture window;
determine one or more signal-acquisition condition metrics characterizing technical capture quality;
determine whether one or more thresholds are satisfied;
responsive to threshold satisfaction, preprocess at least a portion of the sensor data and extract a plurality of digital biomarker signs;
generate sign-state data for at least one of the plurality of digital biomarker signs;
perform rule-out analysis and rule-in analysis using at least the sign-state data to determine one or more health insights associated with the user;
rank-order the one or more health insights; and
output at least one ranked health insight.

16. The system of claim 15, wherein the instructions are executable in a configuration selected from:
an on-device configuration in which measurement and interpretation are both performed on the user device;
a cloud configuration in which at least part of interpretation is performed by one or more remote computing systems; and
a hybrid configuration in which one or more functions are performed on the user device and one or more other functions are performed by the one or more remote computing systems.

17. The system of claim 15, wherein the instructions further cause the system to generate at least two output renderings from a common underlying inference result comprising sign-state data and one or more health insights, the at least two output renderings comprising a user-facing summary and a clinician-style or structured summary.

18. The system of claim 15, wherein the instructions further cause the system to selectively present, transmit, or export at least one of the plurality of digital biomarker signs, the sign-state data, the one or more ranked health insights, a composite health score, or any combination thereof to at least one of a user-facing interface, a remote computing system, a workflow destination, or a structured output destination.

19. The system of claim 15, wherein the plurality of digital biomarker signs comprise one or more mobility signs selected from walking speed, step length, cadence, double support time, gait variability, asymmetry, balance-related movement, tremor-related movement, one or more longitudinal indices derived therefrom, or any combination thereof.

20. A non-transitory computer-readable medium storing instructions which, when executed by one or more processors of one or more computing systems, cause the one or more processors to:
obtain sensor data during at least one capture window;
determine one or more signal-acquisition condition metrics characterizing technical capture quality;
determine whether the one or more signal-acquisition condition metrics satisfy one or more thresholds;
responsive to determining that the one or more thresholds are satisfied, preprocess at least a portion of the sensor data and extract a plurality of digital biomarker signs;
generate sign-state data for at least one of the plurality of digital biomarker signs;
perform rule-out analysis and rule-in analysis using at least the sign-state data to determine one or more health insights associated with a user;
rank-order the one or more health insights; and
output at least one ranked health insight.
